(19) United States
(12) Patent Application Publication
(10) Pub. No.: US 2007/0043085 A1
(43) Pub. Date: Feb. 22, 2007

(54) Process for the preparation of amorphous form of neutral esomeprazole

(75) Inventors: Bobba Venkata Siva Kumar, Navi Mumbai (IN); Pravin Bhalchandra Kulkarni, Kalyan (IN); Anil Ganpat Suryawanshi, Thane (IN); Changdev Namdev Raut, Navi Mumbai (IN); Nitin Sharad Chandra Pradhan, Thane (IN)

Correspondence Address: M. CARMEN & ASSOCIATES, PLLC, 170 OLD COUNTRY ROAD, SUITE 400, MINEOLA, NY, 11501, US

(73) Assignee: Glenmark Pharmaceuticals Limited, Mumbai (IN)

(21) Appl. No.: 11/507,284

(22) Filed: Aug. 21, 2006

(30) Foreign Application Priority Data
Aug. 19, 2005 (IN) 979/MUM/2005

(51) Int. Cl. A61K 31/4439; C07D 403/02

(52) U.S. Cl. 514/338; 546/273.7

(57) ABSTRACT

A process for preparing neutral esomeprazole in an amorphous form is provided comprising (a) providing an aqueous solution comprising a salt of esomeprazole; (b) neutralizing the solution with a neutralization agent to provide a neutralized solution; (c) contacting the neutralized solution with an extracting solvent; and (d) recovering the neutral esomeprazole in an amorphous form.

### PRIORITY

This application claims the benefit under 35 U.S.C. § 119 to Indian Provisional Application 979/MUM/2006, filed on Aug. 19, 2005, and entitled“PROCESS FOR THE PREPARATION OF AMORPHOUS ESOMEPRAZOLE BASE”, the contents of which are incorporated by reference herein.

### BACKGROUND OF THE INVENTION

1. Technical Field

The present invention generally relates to a process for the preparation of an amorphous form of neutral esomeprazole.

2. Description of the Related Art

The compound 5-methoxy-2-[[(4-methoxy-3,5-dimethyl-2-pyridyl)methyl]sulfinyl]-1H-benzimidazole, having the generic name omeprazole, and therapeutically acceptable salts thereof are well known as gastric acid secretion inhibitors, and are useful as anti-ulcer agents. The compound, being a sulfoxide, has an asymmetric center in the sulfur atom and may exist as a racemic mixture of its two single enantiomers, the R-omeprazole and the S-omeprazole. The optical isomers of omeprazole, particularly the (S) isomer, are believed to possess certain advantages over the racemic form. The absolute configurations of the enantiomers of omeprazole have been determined by an X-ray study of an N akylated derivative of the (+)-enantiomer in neutral form. The (+)-enantiomer of the neutral form and the (−)-enantiomer of the neutral form were found to have the R and S configuration, respectively. The conditions for the optical rotation measurement for each of these enantiomers are described in WO 94/27988.

The enantiomer (S)-omeprazole is commonly referred to as esomeprazole (also known as (5-methoxy-2-[(S)-[(4-methoxy-3,5-dimethyl-2-pyridyl)methyl]-sulfinyl]-1H-benzimidazole-1-yl) and is represented by the structure of Formula I. 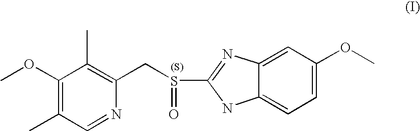
Esomeprazole is marketed in the United States as the magnesium trihydrate salt under the name Nexium® and is indicated for short-term treatment in the healing and symptomatic resolution of diagnostically confirmed erosive esophagitis. See, e.g., The Merck Index, Thirteenth Edition, 2001, pp. 1224-25, monograph 6913; and Physician's Desk Reference, “Nexium,” 58th Edition, pp. 624-28 (2004).

Other compounds from this class of compounds which are commercially available or in clinical trials are racemic mixtures of pantoprazole, lansoprazole, rabeprazole and tenatoprazole.

U.S. Pat. No. 5,693,818, incorporated by reference herein, discloses processes for the preparation of esomeprazole. In one process for the preparation of esomeprazole, a substituted 2-(2-pyridinylmethylsulfinyl)-1H-benzimidazole is resolved to the corresponding enantiomers followed by hydrolysis.

U.S. Pat. No. 5,948,789, incorporated by reference herein, also discloses a process for preparing esomeprazole including a selective oxidation. This process includes oxidizing the sulfur atom and resolving the enantiomers in one step.

Certain optically pure salts of (R) and (S)-omeprazole are described, for example, in U.S. Pat. No. 5,714,504 and European Patent 124,495. The optical isomers of omeprazole, particularly the (S) isomer, are believed to possess certain advantages over the racemic form. For example, the optically pure salts of omeprazole disclosed in International Publication No. WO 94/27988 are said to have an improved therapeutic profile such as lower degree of interindividual variation.

A number of drugs have been found to exhibit desirable dissolution characteristics and, in some cases, desirable bioavailability patterns when used in a specific solid form, e.g., as an amorphous or crystalline solid. Therefore, there is a continuing need for new solid forms of esomeprazole and/or process for their preparation.

### SUMMARY OF THE INVENTION

In accordance with one embodiment of the present invention, a process for preparing neutral esomeprazole in an amorphous form is provided comprising (a) providing an aqueous solution comprising a salt of esomeprazole; (b) neutralizing the solution with a neutralization agent to provide a neutralized solution; (c) contacting the neutralized solution with an extracting solvent; and (d) recovering the neutral esomeprazole in an amorphous form.

In accordance with a second embodiment of the present invention, neutral esomeprazole in an amorphous form prepared by the process comprising (a) providing an aqueous solution comprising a salt of esomeprazole; (b) neutralizing the solution with a neutralization agent; (c) contacting the neutralized solution with an extracting solvent; and (d) recovering the neutral esomeprazole in an amorphous form is provided.

In accordance with a third embodiment of the present invention, a pharmaceutical composition is provided comprising a therapeutically effective amount of neutral esomeprazole in an amorphous form.

In accordance with a fourth embodiment of the present invention, a method for reducing gastric acid secretion in a subject is provided which comprises administering to the subject an amount of neutral esomeprazole in a solid amorphous form and effective to reduce gastric acid secretion by the subject.

The advantages of the process of the present invention include simplicity, eco-friendliness and suitability for commercial use.

Definitions

The term “treating” or “treatment” of a state, disorder or condition as used herein means: (1) preventing or delaying the appearance of clinical symptoms of the state, disorder or condition developing in a mammal that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the patient or to the physician.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host means causing a therapeutically effective blood concentration of the active ingredient at the particular location. This can be accomplished, e.g., by topical, local or by systemic administration of the active ingredient to the host.

The term “subject” or “a patient” or “a host” as used herein refers to mammalian animals, preferably human.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a preparation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in tablet granulations. Such compounds include, by way of example and without limitation, acacia alginic acid, tragacanth, carboxymethylcellulose sodium, poly (vinylpyrrolidone), compressible sugar (e.g., NuTab), ethylcellulose, gelatin, liquid glucose, methylcellulose, povidone and pregelatinized starch, combinations thereof and other material known to those of ordinary skill in the art.

When needed, other binders may also be included in the present invention. Exemplary binders include starch, poly(ethylene glycol), guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and other such materials known to those of ordinary skill in the art.

The term “diluent” or “filler” as used herein is intended to mean inert substances used as fillers to create the desired bulk, flow properties, and compression characteristics in the preparation of tablets and capsules. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in tablet and capsule formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in tablet formulations to reduce friction during tablet compression. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage forms to promote the disruption of the solid mass into smaller particles which are more readily dispersed or dissolved. Exemplary disintegrants include, by way of example and without limitation, starches such as corn starch, potato starch, pre-gelatinized and modified starched thereof, sweeteners, clays, such as bentonite, microcrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxyl propylcellulose, hydroxypropylmethylcellulose phthalate, noncrystalline cellulose, magnesium aluminum silicate, triethanolamine, polyvinyl alcohol, polyvinylpyrrolidone (PVP), tyloxapol (a nonionic liquid polymer of the alkyl aryl polyether alcohol type, also known as superinone or triton), combinations thereof and other such materials known to those of ordinary skill in the art.

Most of these excipients are described in detail in, e.g., Howard C. Ansel et al., Pharmaceutical Dosage Forms and Drug Delivery Systems, (7th Ed. 1999); Alfonso R. Gennaro et al., Remington: The Science and Practice of Pharmacy, (20th Ed. 2000); and A. Kibbe, Handbook of Pharmaceutical Excipients, (3rd Ed. 2000), which are incorporated by reference herein.

### BRIEF DESCRIPTION OF THE DRAWING

FIG. 1 is a characteristic X-ray powder diffraction pattern of neutral esomeprazole in an amorphous form.

### DETAILED DESCRIPTION OF THE INVENTION

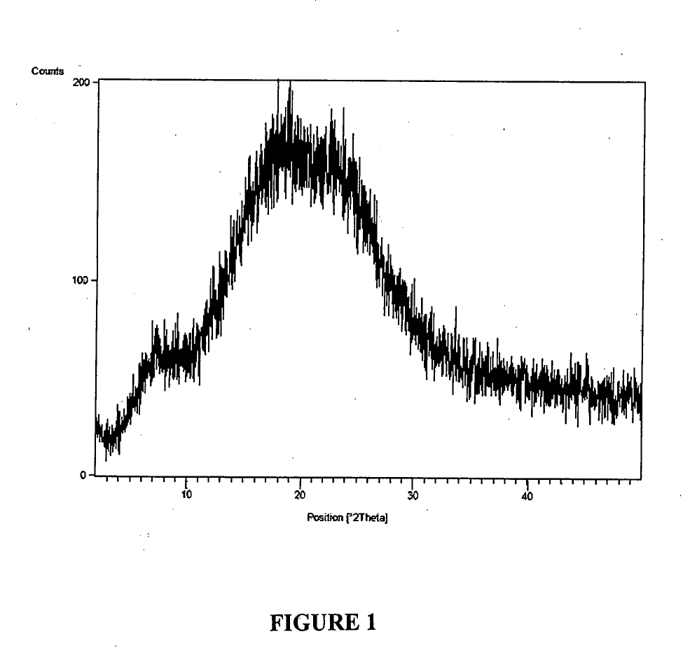

The present invention is directed to an improved process for the preparation of neutral esomeprazole, i.e., not in the form of a salt, in an amorphous form. The amorphous form of amorphous esomeprazole can be characterized by X-ray powder diffraction (XRD) pattern. Generally, amorphous materials do not exhibit the three dimensional long range order found in crystalline materials, but are structurally more similar to liquids where the arrangement of molecules is random. The XRD pattern of the amorphous esomeprazole in an amorphous form of the present invention is substantially in accordance with FIG. 1. The x-ray powder diffractogram was measured on a Xpert pro analytical diffractometer.

In one embodiment, a process of the present invention may be carried out by at least (a) providing an aqueous solution containing at least a salt of esomeprazole, (b) neutralizing the solution with a neutralization agent, (c) contacting the neutralized solution with an extracting solvent, and (d) recovering the neutral esomeprazole in an amorphous form.

In step (a) of the process of the present invention, the aqueous solution of esomeprazole salt may be obtained by dissolving a suitable esomeprazole salt, e.g., a water soluble salt of esomeprazole, in a suitable solvent. Suitable salts of esomeprazole include, but are not limited to, alkaline salts of esomeprazole, such as esomeprazole sodium salt and esomeprazole potassium salt, and the like. The salts of esomeprazole for use as a starting material may be in the form of any of the various polymorphic or hydrate forms known in the art. Esomeprazole salts such as esomeprazole sodium and esomeprazole potassium may be prepared by any of the known methods, see, e.g., U.S. Pat. Nos. 4,738,974 and 5,693,818, the contents of which are incorporated by reference herein. A suitable solvent for use in this step includes, for example, water and the like and mixtures thereof.

In step (b) of the process of the present invention, a suitable neutralization agent is added to the solution prepared in step (a) to neutralize the solution. The neutralization agent will be added in an amount sufficient to neutralize the solution to a pH of about 7 to about 11 and preferably from about 9 to about 11.

In one embodiment, the neutralization agent can be an acid-base salt. Suitable acid-base salts include, but are not limited to, ammonium acetate, ammonium chloride, ammonium bromide, pyridine acetate, triethylamine acetate, and mixtures thereof.

In another embodiment, the neutralization agent can be a suitable acid. Suitable acids include, but are not limited to, hydrochloric acid; sulfonic acid; sulfuric acid; sulfurous acid; phosphoric acid; carbonic acid; saturated or unsaturated C₁-C₄ unsubstituted or halo- or hydroxy-substituted alkanoic mono and di-carboxylic acids such as formic acid, acetic acid, propionic acid, citric acid, tartaric acid, maleic acid, oxalic acid, chloroacetic acid and the like; arylalkanoic acids such as benzoic acid and the like; alkylsulfonic acids such as methanesulfonic acid and the like; aryl sulfonic acids such as p-toluene sulfonic acid and the like; and mixtures thereof. A preferred acid is an acid with a pKa of about 4 to about 6.

In step (c) of the process of the present invention, the neutralized solution is contacted with an extracting solvent. Suitable extracting solvents for use herein include chlorinated solvents and the like and mixtures thereof. Useful chlorinated solvents include, but are not limited to, chloroform, carbon tetrachloride, perchloroethylene, methylene chloride and the like and mixtures thereof. Preferably, the extracting solvent is methylene chloride.

The neutral esomeprazole may be recovered from the extracted solution by concentrating the organic phase from the extraction. For example, the extracting solvent may be removed by heating, preferably under reduced pressure using, for example, a rota evaporator, to provide a solid residue of the neutral esomeprazole in an amorphous form. The solution can be heated to a temperature sufficient to remove the extracting solvent, e.g., a temperature ranging from about 0° C. to about 60° C., preferably from about from about 20° C. to about 45° C., and most preferably from about from about 30° C. to about 40° C.

The solid residue obtained after the solvent removal may be isolated and dried using conventional methods to provide solid amorphous neutral esomeprazole. For example, the neutral esomeprazole can be slurried with a suitable solvent, and filtered to provide the amorphous form of neutral esomeprazole. Suitable solvents include, but are not limited to, aliphatic hydrocarbons such as n-hexane, isooctane, n-heptane, and the like and mixtures thereof. Preferably, the neutral esomeprazole is concentrated prior to being slurried and filtered. Alternatively, a suitable solvent may be added to the residue followed by evaporation using, for example, a rota evaporator under vacuum, to provide solid amorphous neutral esomeprazole. Suitable solvents include, but are not limited to, ketones such as acetone, methyl ethyl ketone and the like and mixtures thereof. The preferred solvent is acetone.

Yet another aspect of the present invention is directed to pharmaceutical compositions containing at least the novel amorphous form of neutral esomeprazole disclosed herein. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes. Oral dosage forms include, but are not limited to, tablets, pills, capsules, troches, sachets, suspensions, powders, lozenges, elixirs and the like. The novel amorphous form of neutral esomeprazoledisclosed herein also may be administered as suppositories, ophthalmic ointments and suspensions, and parenteral suspensions, which are administered by other routes. The dosage forms may contain the novel amorphous form of neutral esomeprazoledisclosed herein as is or, alternatively, may contain the amorphous form of neutral esomeprazole disclosed herein as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

Capsule dosages will contain the novel amorphous form of neutral esomeprazole disclosed herein within a capsule which may be coated with gelatin. Tablets and powders may also be coated with an enteric coating. The enteric-coated powder forms may have coatings comprising phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric-coating.

Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants such as magnesium and calcium stearate and sodium stearyl fumarate; flavorings; sweeteners; preservatives; pharmaceutically acceptable dyes and glidants such as silicon dioxide.

In one embodiment, the amorphous form of neutral esomeprazole disclosed herein for use in the pharmaceutical compositions of the present invention can have a D₅₀ and D₉₀ particle size of less than about 400 microns, preferably less than about 200 microns, more preferably less than about 150 microns, still more preferably less than about 50 microns and most preferably less than about 15 microns. The particle sizes of the novel amorphous form of neutral esomeprazole can be obtained by, for example, any milling, grinding, micronizing or other particle size reduction method known in the art to bring the solid state amorphous form of neutral esomeprazole into any of the foregoing desired particle size range.

Actual dosage levels of the amorphous form of neutral esomeprazole of the invention may be varied to obtain an amount of the amorphous form of neutral esomeprazole that is effective to obtain a desired therapeutic response for a particular composition and method of administration for treatment of a mammal. The selected dosage level therefore depends upon such factors as, for example, the desired therapeutic effect, the route of administration, the desired duration of treatment, and other factors. The total daily dose of the amorphous form of neutral esomeprazole of this invention administered to a host in single or divided dose and can vary widely depending upon a variety of factors including, for example, the body weight, general health, sex, diet, time and route of administration, rates of absorption and excretion, combination with other drugs, the severity of the particular condition being treated, etc.

Further, the amorphous neutral esomeprazole dosage forms described herein can be used in a method for treatment of gastric acid related diseases. The method of treatment includes administering to a mammal in need of treatment a dosage form that includes a therapeutically effective amount of the amorphous form of neutral esomeprazole.

The following examples are provided to enable one skilled in the art to practice the invention and are merely illustrative of the invention. The examples should not be read as limiting the scope of the invention as defined in the features and advantages.

### EXAMPLE 1

Preparation of solid neutral esomeprazole in an amorphous form.

Sodium salt of esomeprazole (5 g) was dissolved in water (250 ml) at room temperature and treated with an ammonium acetate solution (5%). The aqueous layer was further extracted with methylene chloride (50 ml). The organic layer was separated off and evaporated under vacuum at 30° C. to 40° C. Methylene chloride was evaporated on a rota evaporator under vacuum until an almost dry amorphous substance was obtained, which was slurried in n-heptane, filtered and dried under vacuum to provide solid neutral esomeprazole in an amorphous form (3.5 g).

### EXAMPLE 2

Preparation of solid neutral esomeprazole in an amorphous form.

Potassium salt of esomeprazole (5 g) was dissolved in water (250 ml) at room temperature and treated with an ammonium chloride solution (5%). The aqueous layer was further extracted with methylene chloride (60 ml). The organic layer was separated off and evaporated under vacuum at 30° C. to 40° C. Methylene chloride was evaporated on a rota evaporator under vacuum until an almost dry amorphous substance was obtained, which was slurried in n-hexane, filtered and dried under vacuum to provide solid neutral esomeprazole in an amorphous form (3.3 g).

### EXAMPLE 3

Preparation of solid neutral esomeprazole in an amorphous form.

Sodium salt of esomeprazole (5 g) was dissolved in water (250 ml) at room temperature and treated with acetic acid (0.74 g) until a pH of 9-10 was reached. The aqueous layer was further treated with methylene chloride (100 ml). The organic layer was separated off and evaporated under vacuum at 30° C. to 40° C. to obtain a concentrated solution. Acetone (50 ml) was added to the solution and evaporated on a rota evaporator under vacuum until an almost dry amorphous substance was obtained as neutral esomeprazole in an amorphous form (3.5 g).

### EXAMPLE 4

Preparation of solid neutral esomeprazole in an amorphous form.

Potassium salt of esomeprazole (5 g) was dissolved in water (250 ml) at room temperature and treated with acetic acid (0.7 g) until a pH of 9-10 was reached. The aqueous layer was further treated with methylene chloride (100 ml). The organic layer was separated off and evaporated on a rota evaporator under vacuum at 30° C. to 40° C. to obtain a concentrated solution. Acetone (50 ml) was added to the concentrated solution and evaporated on a rota evaporator under vacuum until an almost dry amorphous substance was obtained as neutral esomeprazole in an amorphous form (3.3 g).

### EXAMPLE 5

Preparation of solid neutral esomeprazole in an amorphous form.

Sodium salt of esomeprazole (5 g) was dissolved in water (25 ml) at room temperature and the pH of the aqueous layer was adjusted to neutral by adding acetic acid (0.75 g). The aqueous suspension was extracted with methylene chloride (50 ml). The organic layer was separated and evaporated on a rota evaporator under vacuum to obtain a highly concentrated solution. Acetone (50 ml) was added to the highly concentrated solution and the solvent was again evaporated on a rota evaporator under vacuum until no more solvent distilled out. The solid material was dried under vacuum to provide enantiomerically pure solid neutral esomeprazole in an amorphous form (2.5 g).

While the above description contains many specifics, these specifics should not be construed as limitations of the invention, but merely as exemplifications of preferred embodiments thereof. Those skilled in the art will envision many other embodiments within the scope and spirit of the invention as defined by the features and advantages appended hereto.

What is claimed is:

1. A process for preparing neutral esomeprazole in an amorphous form, the process comprising (a) providing an aqueous solution comprising a salt of esomeprazole; (b) neutralizing the solution with a neutralization agent to provide a neutralized solution; (c) contacting the neutralized solution with an extracting solvent; and (d) recovering the neutral esomeprazole in an amorphous form.

2. The process of claim 1, wherein the salt of esomeprazole is esomeprazole sodium or esomeprazole potassium.

3. The process of claim 1, wherein step (a) comprises dissolving esomeprazole sodium or esomeprazole potassium in water.

4. The process of claim 1, wherein the neutralization agent is an acid-base salt.

5. The process of claim 4, wherein the acid-base salt is selected from the group consisting of ammonium acetate, ammonium chloride, ammonium bromide, pyridine acetate, triethylamine acetate and mixtures thereof.

6. The process of claim 1, wherein the neutralization agent is a suitable acid.

7. The process of claim 6, wherein the suitable acid is selected from the group consisting of hydrochloric acid, sulfonic acid, sulfuric acid, sulfurous acid, phosphoric acid, carbonic acid, formic acid, acetic acid, propionic acid, citric acid, tartaric acid, maleic acid, oxalic acid, chloroacetic acid, benzoic acid and mixtures thereof.

8. The process of claim 1, wherein the extracting solvent is a chlorinated solvent.

9. The process of claim 1, wherein the extracting solvent is a chlorinated solvent selected from the group consisting of chloroform, carbon tetrachloride, perchloroethylene, methylene chloride and mixtures thereof.

10. The process of claim 1, further comprising removing the extracting solvent from the solution to form a solid residue.

11. The process of claim 10, wherein the extracting solvent is removed by heating at an elevated temperature under reduced pressure.

12. The process of claim 10, further comprising adding an aliphatic hydrocarbon solvent to the solid residue and filtering the neutral esomeprazole in an amorphous form.

13. The process of claim 12, wherein the aliphatic hydrocarbon solvent is selected from the group consisting of n-hexane, isooctane, n-heptane and mixtures thereof.

14. An amorphous form of neutral esomeprazole prepared by the process of claim 1.

15. The amorphous form of neutral esomeprazole of claim 14, further characterized by having an X-ray diffraction pattern substantially in accordance with FIG. 1.

16. A pharmaceutical composition comprising a therapeutically effective amount of the amorphous form of neutral esomeprazole of claim 14 and one or more pharmaceutically acceptable carriers.

17. The pharmaceutical composition of claim 16, in the form of a solid.

18. A method for reducing gastric acid secretion in a subject which comprises administering to the subject an amount of the amorphous form of neutral esomeprazole of claim 14 in solid form and effective to reduce gastric acid secretion by the subject.

19. A process for preparing neutral esomeprazole in an amorphous form, the process comprising (a) providing an aqueous solution comprising a salt of esomeprazole; (b) adding an acid-base salt to the solution; (c) contacting the solution of step (b) with a chlorinated solvent; and (d) recovering the neutral esomeprazole in an amorphous form from the solution of step (c).

20. The process of claim 19, wherein the acid-base salt is selected from the group consisting of ammonium acetate, ammonium chloride, ammonium bromide, pyridine acetate, triethylamine acetate and mixtures thereof.

21. The process of claim 19, wherein the chlorinated solvent selected from the group consisting of chloroform, carbon tetrachloride, perchloroethylene, methylene chloride and mixtures thereof.

22. The process of claim 19, wherein the step (d) comprises heating the solution of step (c) to a temperature sufficient to substantially remove the chlorinated solvent and obtain a solid residue; adding a suitable solvent to the solid residue followed by filtration to provide the neutral esomeprazole in an amorphous form.

23. The process of claim 22, wherein the solution is heated to a temperature of about 20° C. to about 45° C.

24. The process of claim 22, wherein the suitable solvent is selected from the group consisting of n-hexane, isooctane, n-heptane and mixtures thereof.
